SEQUENCE LISTING (6 sequences)

SEQ ID NO: 1 - DNA, 6968 bp, Eschscholzia californica
aaatttagttgcaaattcaatttttctaatttcttttctgttgctatatgattcataagtgttagaaaatgattccatatatgtattaggcgtggtcgtcgtaaagtttttggaattttgtaaaccaaaaattactatatataagttagatattctccaaaagtgtctgatttatatgaaaagaataattgaaaacaagattttataacaactaactcccaagaattacatataaaaattcaagattatggacaagtaattgatgacaactatcttcaacaaaaaaaaaaaattgatgacaactaacttcccttagttgtctttgtggagtagttgtttgacaagtgaaaatgtaagatggtgattggcaagttattgatggcaaccaacttgttacttttccggtggtagctgtcaattgcccttccattagggctcgtcagtgttataggtattcatggggacatctaacatgtacatagaccctagtatccttccataattttatgattgattgattctatcctctcattaagtctatataaggctaatactggtttcccattctgttggctactatcaaaatgacctaattagcaaagacaatttatctagttgactactatactaacaatggaactccctaaccaaagaaagagaagatatgggtgggtggggggagagtgacattcttctctaggttttcaattcctcccttgatcttaattttcctttctttcttttattgttttagattttcattatcacaagtaatcaacttgagttttcaacaaaaaaaaataatcaacttgagtataaaatttaatataaaatattagattttaattaataatcaactacaaaatttgagcgggacaaataaaaatcacgtgatttttttttataaaaggcaattaaacgttgtctgtaatcatgtaagaaagattctgactgaatgtataaaaaaattaatagttaagacgtggaaagaaccaaaaaccgtagtatatagtaattaattattttataaggtccaaattattattattagtcaatctaaccaggctgtattactattactaaatctattaatttatgaaaaattagctcgtaatctctttcttcccaaatttactcccacgcacacctcgtggtaggtcatcgtgcgtctcttatctattaactcatttgtttttaaatccctaagtcacctttataatcctgattttctaatccaacggtgcgtaattctctaacttgatcttgaagtccgcatctcattcttagaaccactgattgaaatatttgggatttgtttcgggatttgaccggtctcaataatagatttgtggacagacagccgccaatctggacagtatcttacacgtgtgagatgactgtaggatatgtggggtccatagaattagttgaccaaatcaaagttagttgacttaaaattcacagtataaataggattgacaatcataactggcattctcgtagtaaaagaaagaaaaaaagaaaaacccccatggaaaacaaaactcccatcttcttctctttgtcaatatttctttctttgctaaactgtgccctggggggtaatgatctcctttcttgtttgacctttaatggggttcgtaatcatactgtattttctgcggattcggactcggatttcaaccgattccttcatttgtcaatccaaaacccactgtttcaaaattcattgatttcgaaaccgtcggcgattatattaccgggaagcaaagaagagttatctaataccattagatgtattagaaaaggatcatggactataagattaagaagtggtggtcatagttatgaaggattatcttacacttctgatacaccctttattcttattgatttaatgaatcttaatcgagtttcaattgatcttgaatctgaaacagcttgggttgaatcgggttcaacgcttggtgagctttattatgcgattactgagtcaagtagtaaactcggatttacggctggttggtgtccaaccgttggtactgggggtcatattagtggtggtggttttggtatgatgtcaagaaaatatggtctagcagctgataacgttgtcgatgcaattcttatagacgctaacggtgcgattctagaccgccaagccatgggagaagatgttttttgggctattcgtggtggtggaggtggtgtctggggtgcaatttatgcatggaaaataaaattattacctgtccccgaaaaggtgacggtctttcgtgtgacaaaaaatgtggcaatagatgaagctacaagtttgctacataagtggcaatttgttgcagaagaattagaagaagattttactttatccgtcctcggtggtgcggatgagaaacaagtgtggttgacaatgttagggtttcatttcggactgaaaaccgttgcaaaaagcacgttcgatctattgtttcctgaattagggttggttgaggaagattatctagaaatgagttggggggagtcttttgcttacttagcaggattagaaacagtttctcaactaaataataggttcttgaaatttgatgagagagcttttaaaacaaaagttgatttaaccaaagaaccattgccatcaaaagcgttttatggtttattggaaagattatcaaaagagccaaatgggtttattgctttgaatggatttggaggccaaatgagtaaaattagtagtgattttacgccgtttcctcatcgaagtggtacaagattaatggttgaatatatagttgcctggaatcaaagtgaacaaaaaaagaaaaccgaatttttggattggttagaaaaagtatatgaatttatgaaaccatttgtttcaaagaatccaagacttgggtatgttaatcatattgatcttgatcttggagggatagattgggggaataaaactgttgttaataatgccattgagattagtaggagttggggtgagagttattttttatcaaattatgaacgtttaattagggctaagaccttgattgatccaaataatgtatttaatcatccacaaagtatccctccaatggcaaattttgattatttggagaagactttggggagtgatggtggagaagttgtaatatagaatcatcacatagctatataaaaaatttatgttttcataatgtgctttataatctttttggagatatacaatctctaatttttttttttttttgctttattttcattttaatgtttgattattatcatttaaattttctatagctcttttagggagttcaattatttttaaaaaaattcatccttgaaaaagaaaatgtaatcctataaatattctaatagtttagataagataggtaataacagtaccccatactcttttatctctctccgacattatttatgtatgacatccttccaatattttagtttctttggttggattctttgacaaaaaatttgatgagatttctagctaaacgttaaaacaaaaaaatctaaaaaaaactccctatttaactaaatgtattttcgaagtacacaaaacgccctttggttagtatataagaatagaaaatacatacatactaatattaattacattctaaaggataagaggaagcctaaatgtaattttgatccttgtcatttgtgattaacttggcgtgattatatcacgatcaactttttttttttttgaactttatcccgatcaactttttttttttgttaagaattatcacgatcaacttggttcgggtctctttctacctttttttggagaaggaacttgcataaagtgaaatgtttgaatgaggtcaaatgggaccgacttttgaggggcactaaaggactactctatccactctactgaacaaacaaacctgttatatatatgcatacaaacatacatatacatgtagcaatgatccttgtagatttatatactctataaatctacaagatttttgacaaaatgtacaaagaatcattaagtcaaactttgaaaaactttgatcaatttttgtcttttagtgaaaaaaagttattaaatctcaaattttatcaatgatcttgtacatgtagcactcccgtatataaatattacataaattgaggaccttctaattttggggtcctaggcccagaaccggccatgtacatatacatttacatatgtactgaggtcacatgtattggatttgtttagcacaaagtattcaattaatttaatttgtgttgaccaacctaagttttgaggctaaatcctttcacttcatcaaataagacaaagcataaattatcacatagaccttattgtctttgctctggtttcccttcactaaagaagacactataataatgaatgttaatggcgatggcgacagttgccaaaggggttatcttggacagccttcattttcatttggaatggtcgtcgccaatagccactttgtgtggcttggataagcaactcctgacaagttcacctatggtcatctacattgatgtcattaatagacatttcaatgtcgtcggctaagtgtcccttcgctgacggcgttaggaaagtcgtgggcactacttttcaccgcaagttttggttaaagagtttcacgttaaaagtaaagaaaaattcacagtaaacggaagttattgtccccaccttcgatgcatgggcggaactattagctgcccggcctggtctccacaaatataactaaatctaccactgattactgtggtttactgttagctctagtggtgaaggccatatgcttccaacccacaggcacaagttcaaatcttgctgcctcctccctttggttatttttcattttgaattggttttctttttaattttcatattggttacaattgttaaaaaaatattgtctttcttaggtaatcttgttttgctcgggtacactctcgtttaagtaatattttctacttcaagtgtcattgaatagttataacacgacgactttagattaatgaataataaaattatgtgacttctttgttcatattttagtactcaattggattatccaatgttacgttaatttgtaaatttaagctcaaaatattacttctgttgttttgtttagattgttaagataaatgttgaaatcatgatatgaaaggatgttcttaaaagagttattttgtgttgtatcttgtaaggtcatacgtattatatgtagaagtttactttatagagactttgggcttgccctctctagtattaaatcttggttccgcccctgcttcgatggtgatattttctttggtttgggcacggtaattcggaaaattttatattcgatggtgttatggtttgctgcgttagcgcctaaaaagccctaatgtagaaaaagaaatttctttccttttgtctttggttttctgtcacaactttgctccacctctatgttgttcagatccaccacgacctacctaaatcaacaatgaaacgtttttacaccgtttatatgtagtgaaccaaccttaaatttttgacacttccatttttttgcgttcccttcattaatgaagaaaatcatttgtttttctactggcattactgttttcacttctttaataaaaagacaaaacgtaatttcaacgctagtgttacttacattatttcctttatttcatttaccaaagtgaggcctacgttttaattgctctcacatcacccacagagactttagttttgcacattaaaacattgttgacttctcggaaaaccaacccaattttgacgcccttgttattggaaccacatcattagcaaagaccaaagttacgacgttgatgtcattggctttacttcgatatcaaagactatatacgttactgtctttcacttcatgaacgatgaccaaacttattaagcaaatatttatcttgactccattgatgaataccgattttgatgctgatgttcttgtcttaacttctacctttataaggaacaccaaaggttagtgatcttgaactttgtggatgttaatgtcattgtgttgacctcattaacaaagatcaaatgtaatttaaatgtgagaactttgccttatggaactgtttctttttgggttatttcccaaatgacccccaactttgtgacgagcttcctgcctgaccacctattttagcaaatattcccgcatggctccctttttccgttatctttaactgtccgttactctctctcctccattttctctctctgccaagtcacctatcacggccacatcatcatcttctccggccggcgaccttccaatattaaaaaaaaaaaaattatttttatttttatttttattttctttctctagaaaaaagaaattttttttcctcaccctctctctaaaactccgtcgaaggggtttccctttgccggagctagtctgttcgtgaacagacctagatctgctcatgaaagaccttgaaggtctgttcgtgaacagttcagggttgttctgttcacgaacagacttgatctaggtctgttcaacgaacagactagctccggcgaatgggtttcccttcgccgagttttagagagagagagggtgaggaaaaaaaatgggttatttcccaaatcgcccctaactttgtgaccagcttcctgcctggccccctactttagcaaatattctcgtatggcctccttttctgttatctttaactgttcgttactctctctcctccatctcattttctctctctgccaagccacctatcacgccacatcatcatcttctacggcaggggtcgccgggcggccaaatttccggcgaagggaaacccctttgccggagctaggtctgttcgttgaatagaccttgcaaggtctgtcaacgaacagacctagattgctcacgaacagaccttcaaggtcgccgtgagaaaaaaaaatatatttt

SEQ ID NO: 2 - DNA, 30 bp, Artificial sequence
  Synthetic nucleic acid
atggatccgattcggactcggatttcaacc

SEQ ID NO: 3 - DNA, 29 bp, Artificial sequence
  Synthetic nucleic acid
attaagcttccacttcgatgaggaaacgg

SEQ ID NO: 4 - DNA, 30 bp, Artificial sequence
  Synthetic nucleic acid
aatctcgagattcggactcggatttcaacc

SEQ ID NO: 5 - DNA, 26 bp, Artificial sequence
  Synthetic nucleic acid
cgaattccacttcgatgaggaaacgg

SEQ ID NO: 6 - DNA, 20 bp, Artificial sequence
  Synthetic nucleic acid
gagctacacatgctcaggtt

(19) United States
(12) Patent Application Publication
(10) Pub. No.: US 2007/0298481 A1
(43) Pub. Date: Dec. 27, 2007

(54) Method for Producing a Useful Intermediate in Alkaloid Biosynthesis By Using Rnai Technology

(76) Inventor: Fumihiko Sato, Kyoto (JP)

Correspondence Address: BIRCH STEWART KOLASCH & BIRCH, PO BOX 747, FALLS CHURCH, VA, 22040-0747, US

(21) Appl. No.: 10/572,395

(22) PCT Filed: Sep. 15, 2004
(86) PCT No.: PCT/JP04/13449

§ 371 Date: Mar. 16, 2006

(30) Foreign Application Priority Data
Sep. 17, 2003 (JP) 2003-324960

(51) Int. Cl. C12N 9/00; A01H 5/00; C07H 21/04; C12N 9/10; C12N 9/88; C12N 15/00; C12N 5/04

(52) U.S. Cl. 435/193; 435/183; 435/232; 435/320.1; 435/419; 536/23.2; 800/298

(57) ABSTRACT

The present invention provides a method for producing an intermediate in alkaloid biosynthesis, which comprises: inhibiting the expression of an enzyme that uses said intermediate as its substrate in an alkaloid producing plant cell, plant tissue or plant body by using RNAi technology as well as RNAi gene used for said method.

### TECHNICAL FIELD

The present invention relates to a method for producing a useful intermediate in alkaloid biosynthesis.

### BACKGROUND ART

Alkaloid is the generic term for basic nitrogen-containing compounds contained in plants. Alkaloids are classified into quinoline-, isoquinoline-, indole-, tropane-, xanthin-typed alkaloids and the like according to their main skeletons. Many kinds of alkaloids are known to be pharmaceutically useful. For example, codeine and morphine are known to have analgesic properties and they are commercially valuable. Noscapine is useful because of its antitussive action. Papaverine is used as a smooth muscle relaxant and a cerebral vasodilator. Berberine has been used as a compound with antibacterial activity, antimalarial activity and antipyretic property.

Isoquinoline alkaloid is the generic term for alkaloids which have isoquinoline as the basic skeleton. Isoquinoline alkaloids are widely distributed in nature and have various structures. Examples of isoquinoline alkaloids include morphine type (such as morphine), protoberberine type (such as berberine) and benzophenanthridine type (such as sanguinarine). Examples of plants which produce isoquinoline alkaloids include Papaveraceae, Berberidaceae, Ranunculaceae, Menispermaceae, Rutaceae and the like.

Among the intermediates of the biosynthesis pathway of isoquinoline alkaloids, attention has been focused on reticuline since it is an important precursor for many pharmacological compounds. For example, Patent literature 1 discloses a method for producing reticuline which comprises introducing mutations randomly into the genome of poppy and selecting the mutants with high reticuline content. This method, however, is cumbersome because it comprises steps of selecting the plants with high reticuline content among randomly generated mutants and extracting reticuline from the selected mutants (see Patent literature 1).

Berberine bridge enzyme is an enzyme involved in the isoquinoline alkaloid biosynthesis pathway and it uses reticuline as the substrate. There have been attempts to develop a method to increase the content of reticuline, which is the substrate of berberine bridge enzyme by decreasing the expression level of berberine bridge enzyme by means of genetic engineering. These attempts employed antisense methods in order to specifically inhibit the expression of berberine bridge enzyme. These attempts resulted in the inhibition of berberine bridge enzyme expression and the reduction of the alkaloid content in general. However, no specific accumulation of intermediate including reticuline has been observed (see for example, Nonpatent literatures 1 and 2).

Recently, RNAi technology, a method other than the antisense method, has been employed to suppress gene expression. RNAi technology (RNA interference) is a method that suppresses the expression of a target gene which has a sequence homologous to dsRNA (double-stranded. RNA) by introducing the dsRNA into target cells. RNAi has been employed for analyses of gene functions in a variety of species. For example, Nonpatent literature 3 reported that the specific inhibition of an enzyme involved in the steroid synthesis system by means of RNAi technology resulted in the accumulation of the intermediate which is the substrate of said enzyme. However, according to the literature, there was considerable conversion from the intended intermediate into other compounds, and thus the specific accumulation of the intended intermediate was unsuccessful. The possible reason why complete shut-off of the metabolic pathway could not be achieved is that steroids are essential components of cell membranes and are closely related to the cell growth. The literature also reported that the growth of plant cells was inhibited (for example, see Nonpatent literature 3).

RNAi vector used for RNAi technology is constructed in order to express double-stranded RNA (dsRNA) in a plant body. RNAi vectors are roughly classified into two types depending on their structures.

One of them is a combination of two plasmids constructed independently; the one expresses sense RNA and the other expresses antisense RNA. Cells are transformed with the mixture of these two plasmids to form dsRNA in the cells. The other is a plasmid which expresses RNA with hairpin structure. As for the latter, there are reports demonstrating. RNAi in Caenorhabditis elegans, Drosophila, plants, Trypanosomatidae and the like. With regard to the plant cases, there is a report which compares the gene silencing effect achieved by a construct which has intron introduced in the middle of hairpin structure of a transgene and that achieved by a construct without such introduction of intron. In this study, it is reported that the transgene with intron is more effective in the expression silencing than that without intron.

The present inventors have hitherto found that gene silencing is attained by expressing dsRNA of about 100 base pairs with about 80% homology to the target gene. Upon the silencing, it is thought that dsRNA is degraded by RNAse within the cell to give siRNAs of about 20 base pairs which are incorporated into the complex called as RISC which degrades the target mRNA. In animal cells, it is reported that the introduction of siRNA of about 20 base pairs could silence gene expression.

Patent literature 1: Japanese patent unexamined publication No. 2002-508947

Nonpatent literature 1: Sang-Un Park et al., Plant Physiology, vol. 128, p. 696-706 (February 2002)

Nonpatent literature 2: Sang-Un Park et al., Plant Molecular Biology, vol. 51, p153-164 (2003)

Nonpatent literature 3: Celine Burger et al., Journal of Experimental Botany, vol. 54, No. 388, p1675-1683 (July 2003)

### DISCLOSURE OF INVENTION

### Problems to be Solved by the Invention

An object of the present invention is to provide a method for producing a specific intermediate in alkaloid biosynthesis.

### Means for Solving the Problems

The present invention provides a method for producing an intermediate in alkaloid biosynthesis, which comprises: inhibiting the expression of the enzyme that uses said intermediate as its substrate in an alkaloid producing plant cell, plant tissue or plant body by using RNAi technology.

In particular, the method comprises inhibiting the expression of the enzyme which uses a specific intermediate in alkaloid biosynthesis as its substrate by means of the RNAi gene as described hereinafter which shuts-off the metabolic pathway and causes the accumulation of the target intermediate in alkaloid biosynthesis in plant cells.

The present invention also provides the intermediate in alkaloid biosynthesis produced by the above-described method.

In addition, the present invention provides a gene used for the above method, which comprises:

i) a promoter, and

ii) sequences of a) and b) downstream to the promoter:

a) a forward sequence homologous to the sequence coding for all or a part of the enzyme that uses said intermediate as its substrate,

b) a reverse sequence complementary to said forward sequence.

In the present specification, said gene is called as “RNAi gene”.

Moreover, the present invention provides a combination of genes used for the above method, which comprises genes of A and B:

A. i) a promoter, and

ii) downstream to the promoter, a gene comprising a forward sequence homologous to the sequence coding for all or a part of the enzyme that uses said intermediate as its substrate,

B. i) a promoter, and

ii) downstream to the promoter, a gene comprising a reverse sequence complementary to said forward sequence.

In the present specification, said combination of genes is called as “combination of RNAi genes”.

The term “forward sequence homologous to the sequence coding for all or a part of the enzyme that uses said intermediate as its substrate” means the sequence introduced into constructs such as vector in the same direction as transcription, which is homologous to the sequence coding for all or a part of the enzyme that uses the target intermediate in alkaloid biosynthesis as its substrate, and has length no less than about 100 bp.

The term “sequence coding for all or a part of the enzyme” includes not only the sequence of the translated region of the gene coding for said enzyme, but also that of the untranslated region of the gene.

The term “reverse sequence complementary to said forward sequence” means the sequence which has complementarity to the above-defined “forward sequence homologous to the sequence coding for all or a part of the enzyme that uses said intermediate as its substrate”. In other words, the term refers to the sequence which is introduced into constructs such as vector in the reverse orientation to transcription and which has homology to the forward sequence.

In the RNAi gene, both of the forward sequence and the reverse sequence must be positioned downstream to the promoter, but either of the forward sequence or the reverse sequence may be positioned upstream to the other.

In the RNAi gene, it is preferable that a spacer sequence lies between the forward sequence and the reverse sequence. The interposition of a spacer provides a space which allows an easy pairing of the forward sequence and the reverse sequence (hereinafter, the repeat including the forward sequence and the reverse sequence is called as “inverted repeat”). The spacer sequence is not limited but usually is a sequence of from several hundred base pairs to 1 kb length. For example, an intron sequence is preferably used.

By the expression of RNAi gene comprising a forward sequence, a spacer sequence and a reverse sequence in plant cells, the expression of the target alkaloid biosynthetic enzyme in the plant cells is suppressed.

DNA comprising a forward sequence, a spacer sequence and a reverse sequence complementary to the forward sequence is transcribed into mRNA by the action of promoter in plant cells. The single-stranded RNA transcribed from the forward sequence and the single-stranded RNA transcribed from the reverse sequence form complementary pairing by hydrogen bondings. Preferably, such RNA forms double-stranded RNA (dsRNA) having hairpin structure with a spacer sequence. This dsRNA is thought to bring about RNAi, i.e., the suppression of the expression of the gene of target alkaloid biosynthetic enzyme.

When the combination of RNAi genes of the present invention is used, both of the vector comprising a forward sequence downstream to a promoter (called as a sense vector) and the vector comprising a reverse sequence downstream to another promoter (an antisense vector) are introduced to plant cells. The forward sequence and the reverse sequence are transcribed into mRNAs by the action of the promoters in plant cells, and the single-stranded RNA transcribed from the forward sequence and the single-stranded RNA transcribed from the reverse sequence form complementary pairing by hydrogen bondings to give double-stranded RNA (dsRNA). This dsRNA is thought to bring about RNAi, i.e., the suppression of the expression of the gene of the target alkaloid biosynthetic enzyme.

By using the RNAi gene or the combination of RNAi genes as described above, the resulting double-stranded RNA inhibits the expression of the gene of target alkaloid biosynthetic enzyme and then the intermediate in alkaloid biosynthesis which is the substrate of the enzyme is specifically accumulated in cells.

“Sequence coding for all or a part of the enzyme that uses said intermediate as its substrate” mentioned above may not necessarily be within the coding region of the target gene, but may be a sequence positioned in 5′UTR or 3′UTR region and may be a sequence positioned in the promoter region. RNAi occurs by using such sequences as those of noncoding regions.

Moreover, the present invention provides the vector described above and the plant cell, plant tissue or plant body transformed by the vector.

Advantageous Effect of the Invention

In previous studies, the suppression of accumulation of the end product of a biosynthesis pathway was already reported. However, in these studies, there was no concept of large scale production of an intermediate by means which allow specific accumulation of the intermediate metabolite nor that of production of novel compounds by adding novel reaction pathway as the present invention. If any, there was no technology which pragmatizes such concepts. In the present invention, the inventors have found that a target intermediate metabolite of biosynthesis pathway is accumulated by using RNAi technology. The present invention suggests the possibilities of accumulations of useful metabolites in a variety of metabolic pathways. The present invention has a wide variety of applications.

### BRIEF DESCRIPTION OF DRAWINGS

FIG. 1 shows the biosynthesis pathway and the biosynthetic enzymes of isoquinoline alkaloids.

FIG. 2 shows the biosynthesis pathway and the biosynthetic enzymes of indole alkaloids.

FIG. 3 shows compounds derived from the intermediate reticuline.

FIG. 4 shows compounds derived from the intermediate strictosidine.

FIG. 5 shows the procedure for constructing dsRNA expression vector, pART27-BBEir, which target berberine bridge enzyme (BBE) gene.

FIG. 6 shows LC/MS analysis which indicates accumulation of reticuline (m/z 330) in Eschscholzia californica BBE dsRNA transformants.

FIG. 7 shows BBE enzyme activities of control and BBE dsRNA transformants.

FIG. 8 shows the result of LC/MS which analyzed BBE enzymatic reaction of control and BBE dsRNA transformants.

FIG. 9 shows content of reticuline and sanguinarine in control and BBE dsRNA transformants.

FIG. 10 is a schematic illustration which indicates that the reaction of reticuline into scoulerine is shut-off by the inhibition of BBE.

### DESCRIPTION OF NOTATIONS

1: norcoclaurine-6-O-methyltransferase
2: coclaurine-N-methyltransferase
3: N-methylcoclaurine-3′-hydroxylase
4: berberine bridge enzyme (BBE)
5: glucosidase I/II

### BEST MODE FOR CARRYING OUT THE INVENTION

In the present invention, the target plant, i.e., the plant to which dsRNA is introduced, is not limited and includes any plant which has alkaloid biosynthesis pathway. Preferable alkaloid biosynthesis pathways are isoquinoline alkaloid biosynthesis pathway, indole alkaloid biosynthesis pathway and the like.

Specific examples of plants with alkaloid biosynthesis pathways include, but not limited to, berberine producing plants, for example, Coptis (such as Coptis japonica, Coptis chinensis Franch and Coptis deltoides C. Y. Cheng et Hsiao), Phellodendron (such as Phellodendron amurense), Berberis, Nandina (such as Nandina domestica), Mahonia (such as Mahonia japonica) and Thalictrum (such as Thalictrum minus), morphine-, codeine-, or papaverine-producing plants, for example, Papaveraceae (such as Papaver somniferum Linn, Papaver setigerum DC and Papaver bracteatum), plants which do not produce morphine but produce the closely-related alkaloids, for example, Papaver orientale Linn and Papaver rhoeas, sanguinarine producing plants, for example, Eschscholzia (such as Eschscholzia californica) and Sanguinaria (such as Sanguinaria canadensis L.), corydaline producing plants, for example, Corydalis tuber (Genus Corydalis plants such as Corydalis bulbosa DC., Corydalis ternata Nakai, Corydalis Nakaii Ishidoya, Corydalis decumbens Person), columbamin producing plants, for example, Calumba (Jateorhiza columba), cepharanthine producing plants, for example, Stephania cepharantha, sinomenine producing plants, for example, Sinomenium acutum (such as Sinomenium acutum Rehder et Wilson), emetine producing plants, for example, Cephaelis ipecacuanha and the like.

Among the above, preferable plants are isoquinoline alkaloid producing plants and especially preferable plants are sanguinarine or berberine producing plants such as Eschscholzia, Coptis, Phellodendron, Berberis, Nandina, Mahonia and Thalictrum. The most preferable plant is Eschscholzia californica.

The origin of the target plant to which the RNAi gene is introduced and that of the RNAi gene which is introduced to the plant may be the same or different. Considering the homology between the target gene and the transgene, they are preferably derived from the same plant species.

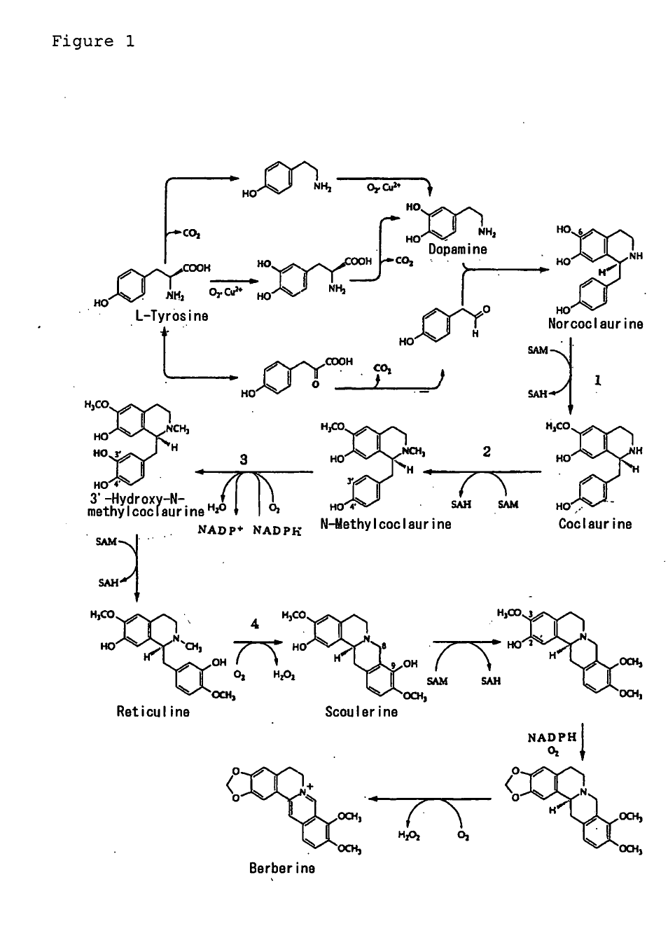

In the method of the present invention, the target to be silenced by RNAi technology is the enzyme which uses the objected biosynthetic intermediate as its substrate. Examples of these enzymes include berberine bridge enzyme (BBE), norcoclaurine-6-O-methyltransferase, coclaurine-N-methyltransferase and N-methylcoclaurine-3′-hydroxylase. All of these enzymes are involved in the isoquinoline alkaloid synthetic pathway (see FIG. 1).

By inhibiting these enzymes by means of RNAi, the substrates of the enzymes, which are the intermediates in the alkaloid biosynthesis, are accumulated. Specifically, by inhibiting berberine bridge enzyme, norcoclaurine-6-O-methyltransferase, coclaurine-N-methyltransferase or N-methylcoclaurine-3′-hydroxylase, reticuline, norcoclaurine, coclaurine, or N-methylcoclaurine is accumulated respectively.

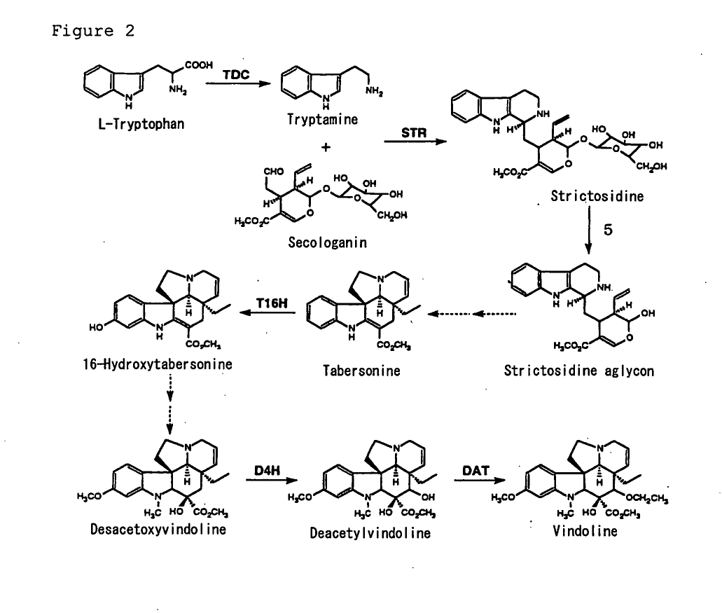

Examples of enzymes other than those in the isoquinoline alkaloid biosynthesis pathway include glycosidase I/II which is an enzyme involved in the indole alkaloid biosynthesis pathway, and by targeting the enzyme, its substrate strictosidine is accumulated (see FIG. 2).

The preferable intermediate in the alkaloid biosynthesis produced by the method of the present invention is selected from the group consisting of reticuline, norcoclaurine, coclaurine and N-methylcoclaurine, and the especially preferable intermediate is reticuline.

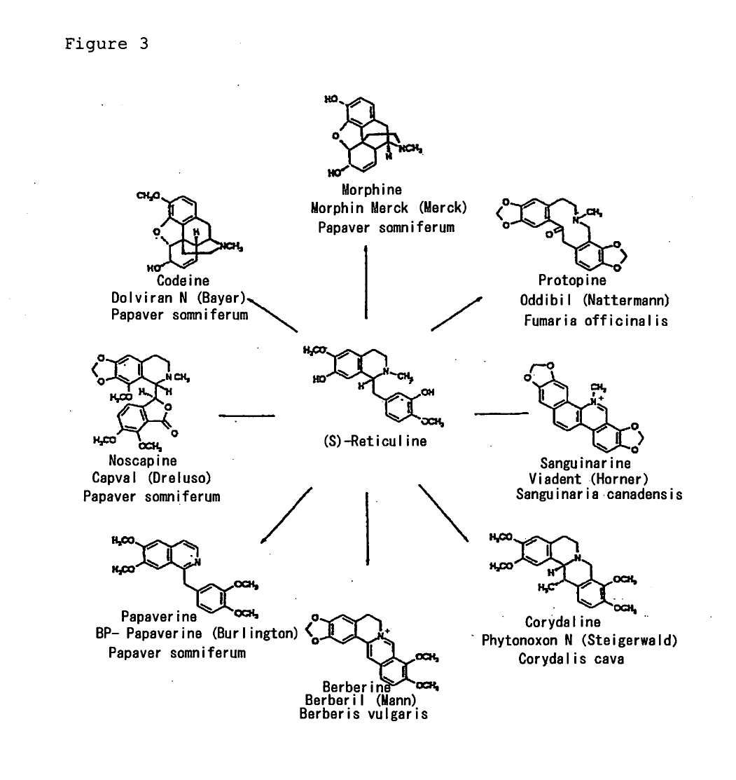

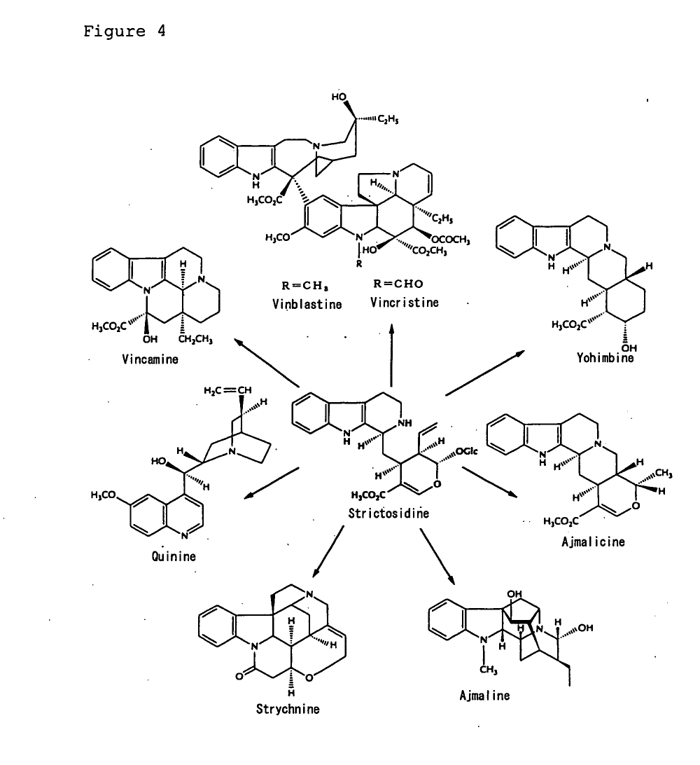

By targeting berberine bridge enzyme, reticuline is accumulated. Reticuline and its precursors such as norcoclaurine, coclaurine and N-methylcoclaurine (see FIG. 1) are useful precursors for various isoquinoline alkaloids shown in FIG. 3. Strictosidine is also useful as a precursor for various indole alkaloids shown in FIG. 4.

In the present invention, preferable RNAi gene which triggers RNAi has a sequence or a part of a sequence which codes for an enzyme selected from the group consisting of berberine bridge enzyme, norcoclaurine-6-O-methyltransferase, coclaurine-N-methyltransferase and N-methylcoclaurine-3′-hydroxylase. The length of the dsRNA that triggers RNAi is not limited, but preferably is no less than 23 bp, more preferably is from 100 bp to 2 kb and the most preferably is from 100 bp to 800 bp.

In the present invention, the promoter which induces expression of the gene that triggers RNAi is not limited as long as it can bring about the expression of the gene when introduced into the target plant. Such promoters are well known to those skilled in the art and include cauliflower mosaic virus 35S promoter, inducible promoters such as alcohol dehydrogenase promoter, tetracycline repressor/operator control system and the like.

In the present invention, homology between forward or reverse sequence and sequence of the gene which codes for the target biosynthetic enzyme may not necessarily be 100%. They may be different in some degrees due to mutation, polymorphism or evolutionary divergence. The dsRNA which has insertion, deletion or point mutation compared to the target gene is also effective in RNAi. The gene used for triggering RNAi may not be completely identical to the target gene, the identity between them is preferably no less than 70%, more preferably no less than 80%, even more preferably no less than 90% and the most preferably no less than 95%.

Similarly, complementarity between forward sequence and reverse sequence is not limited as long as they can form double-stranded RNA after they are transcribed. In order to efficiently form dsRNA, the complementarity between forward sequence and reverse sequence is no less than 70%, preferably at least 80%, more preferably at least 90% and the most preferably at least 95%.

In the present invention, any known method for introducing the vector into the target plant may be employed. Examples of the methods known to those skilled in the art include polyethyleneglycol method, electroporation method, Agrobacterium method, particle bombardment method and the like. The method for preparing vectors and that for regenerating plant bodies from transformed plant cells suited for each of the above methods may be any method known to those skilled in the art depending on the plant species (Toki S, et al., Plant Physiol. 100: 1503, 1995).

Examples of established methods for creating transgenic plants include: introducing a gene to protoplast with polyethyleneglycol and regenerating the plant body (Datta SK: In Gene Transfer To Plants (Potrykus I and Spangenberg, Eds) pp. 66-74, 1995), introducing a gene to protoplast with electrical pulse and regenerating the plant body (Toki S, et al., Plant Physiol. 100: 1503, 1992), introducing a gene to a cell directly by use of particle bombardment and regenerating the plant body (Christou P. et al., Biotechnology 9: 957, 1991), the method which comprises introducing a gene to a cell by use of Agrobacterium and regenerating the plant body (Hiei Y, et al: Plant J 6: 271, 1994) and the like. In the present invention any of the above methods may be suitably employed.

In the present invention, any kind of vectors may be used for introducing RNAi gene into the plants and may be selected depending on the gene transfer method. For example, when Agrobacterium method is used for gene transfer, binary vectors such as pART, pBI101, pBI121 and pIG121Hm are suitably used.

In the present invention, the method for creating the vector is not limited and any well known method may be employed. The vector used for the present invention includes a terminator located 3′ to the transgene. Any known terminator may suitably be used and examples of terminators include OCS terminator, nos terminator, 35S terminator and the like.

In the present specification, identities of nucleotide sequences may be determined by using algorithm of Karlin S & Altschul, BLAST (Proc. Natl. Acad. Sci. USA 87: 2264-2268, 1990, Karlin S & Altschul S F, Proc. Natl. Acad. Sci. USA 90: 5873, 1993). Programs based on BLAST algorithm, such as BLASTN and BLASTX, have been developed (Altschul S F, et al., J Mol. Biol. 215: 403, 1990). The procedures in these analyses are known to the art (http://www.ncbi.mlm.nih.gov/).

### EXAMPLES

The present invention is further described by referring to the specific examples, but it is only for illustrating the present invention and not for limiting the invention.

### Brief summary of examples

The expression vector which produces double-stranded RNA (dsRNA) specifically targeting an alkaloid biosynthetic gene was constructed and owing to its potent silencing effect, the expression of the target gene and the activity of the target enzyme were effectively suppressed. It was confirmed that the intermediate of alkaloid biosynthetic pathway was accumulated.

In particular, the method for producing a biosynthetic intermediate of an isoquinoline alkaloid which may be used as an important pharmaceutical, which comprises shutting-off the metabolic pathway of an alkaloid producing plant cell by RNA interfering (RNAi) technology using double-stranded RNA (dsRNA) is disclosed. The vector which expresses dsRNA corresponding to a part of the sequence coding for berberine bridge enzyme, one of benzophenanthridine alkaloid biosynthesis pathway enzymes, was introduced into Eschscholzia californica cells which produce benzophenanthridine alkaloid. As a result, the alkaloid biosynthetic pathway of Eschscholzia californica was shut-off and reticuline, a biosynthetic intermediate, was accumulated in the plant cells.

### Detailed explanation of examples

The cDNA for berberine bridge enzyme (BBE) was isolated from Eschscholzia californica, which is a transformable plant and produces isoquinoline alkaloid, by using primers designed based on the known sequence of BBE gene isolated from Eschscholzia californica (SEQ ID NO: 1). BBE dsRNA expression vector was constructed based on the cDNA. The vector was introduced to Eschscholzia californica cells to give the transformants expressing the BBE dsRNA. Eschscholzia californica transformants, in which reticuline, a biosynthetic intermediate or the substrate of berberine bridge enzyme, was accumulated, was thus established.

Material and Method

pKANNIBAL and pART27, widely used vectors for the production of inverted repeats, were used for preparing constructs. pKANNIBAL includes CaMV 35S promoter, an intron region which has multiple restriction enzyme sites downstream to the promoter and OCS terminator downstream to the intron region. The forward sequence and the reverse sequence are inserted into both ends of the intron. Thus obtained sequence is transcribed into mRNA in plants and then spliced. Said RNA forms an inverted repeat, i.e., double-stranded RNA (dsRNA). The vector which may be used for the present invention is not limited to the vector used in the examples.

Isolation of BBE gene from Eschscholzia californica

Almost full length of about 1 kb fragment of Eschscholzia californica BBE gene which has BamHI and HindIII restriction sites within its 3′arm (a portion referred as reverse sequence in the present specification) and has EcoRI and XhoI sites within its 5′arm (a portion referred as forward sequence in the present specification) was isolated from cDNA of cultured Eschscholzia californica cells by PCR based on the sequence which had been registered in database.

The primers used for PCR were as follows:

BBE-3′ arm-forward (FW):

(SEQ ID NO: 2)

ATG GAT CCG ATT CGG ACT CGG ATT TCA ACC
reverse (RV):

(SEQ ID NO: 3)

ATT AAG CTT CCA CTT CGA TGA GGA AAC GG
5′ arm-forward (FW):

(SEQ ID NO: 4)

AAT CTC GAG ATT CGG ACT CGG ATT TCA ACC
reverse (RV):

(SEQ ID NO: 5)

CGA ATT CCA CTT CGA TGA GGA AAC GG.

Thus isolated gene was subcloned into plasmid pT7 Blue (Novagen) and sequenced with SIMADZU DSQ-2000L.

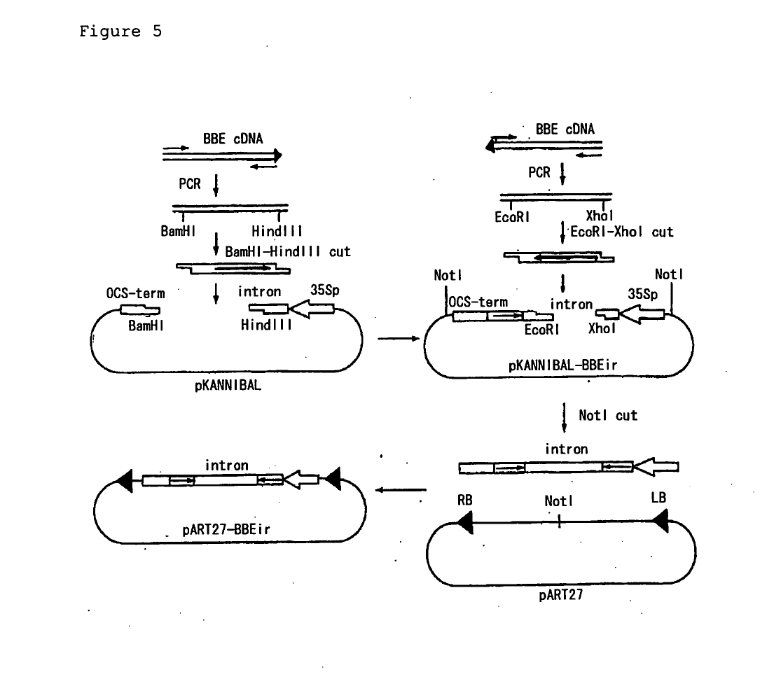

Creation of dsRNA expression vector (FIG. 5) Insertion of 3′arm

PCR product obtained with the primer pair, BBE-3′arm-FW and RV, was subcloned into pT7-Blue, sequenced and digested with BamHI and HindIII restriction enzymes. Vector pKANNIBAL was also digested with BamHI and HindIII. These DNA were electrophoresed, treated with phenol, extracted with chloroform, precipitated with ethanol, dissolved in 10 μl of TE and subjected to ligation reaction. XL1-Blue was transformed with the resulting DNA. The insertion was confirmed by restriction enzyme digestion and sequencing of the OCS terminator with AS1.

Insertion of 5′arm

PCR product obtained with the primer pair, BBE 5′arm FW and RV was subcloned into pT7-Blue, sequenced and digested with EcoRI and XhoI restriction enzymes. In order to avoid tandem insertions, dephosphorylation was carried out with alkaline phosphatase (Calf Intestine. Alkaline Phosphatase: CIAP). In order to inactivate CIAP, the reaction was incubated for 30 minutes at 65° C. Restriction enzymes were then inactivated by ethanol precipitation and DNA was dissolved in 20 μl of TE. The vector pKANNIBAL to which 3′arm had been introduced was also digested with EcoRI and XhoI and subjected to ligation reaction. XL1-Blue was transformed with the resulting vector. The insertion was confirmed by restriction enzyme digestion and sequencing of the OCS terminator with AS1 primer and that of 35S promoter with S1 primer (35Spro-S1, GAG CTA CAC ATG CTC AGG TT (SEQ ID NO: 6). The resulting plasmid to which 3′arm and 5′arm of BBE were inserted was termed as pKANNIBAL-BBEir.

Introduction to binary vector pART27

pART27 vector was digested with restriction enzyme NotI and treated with alkaline phosphatase (Calf Intestine Alkaline Phosphatase: CIAP). The above described plasmid pKANNIBAL-BBEir was digested with NotI to give an insert. Each of thus obtained solution of the vector and that of the insert was extracted with phenol and then with chloroform, and precipitated with ethanol. The vector and the insert were dissolved in 20 μl of TE buffer and the mixture was subjected to ligation reaction. The resulting plasmid was extracted from the obtained colonies, and was digested with restriction enzymes to confirm the insertion of the intended insert. In addition, the resulting plasmid was sequenced by use of 35Spro-S1 as a primer. It was confirmed that the vector pART27-BBEir which expressed the intended dsRNA was created.

Introduction to Agrobacterium

Thus created pART27-BBEir was introduced to Agrobacterium LBA4404 strain by electroporation. The transformation was confirmed by extracting the plasmid from the emerged colonies with Promega SV Minipreps and by digesting the plasmid with restriction enzymes.

Transformation of Eschscholzia californica cells

The expression vector constructed as above was introduced into Eschscholzia californica cells according to the method described in Proc. Nat. Acad. Sci. 98:367-372 (2001)7. Seeds of Eschscholzia californica (California poppy) (Kaneko Seeds, Japan) were wrapped in miracloth and were surface-sterilized with 1% benzalkonium chloride solution for 1 min, 70% (v/v) ethanol for 1 min. and 1% sodium hypochlorite solution for 14 min, and then rinsed three times in sterilized water (each rinse was 5 minutes). Thus sterilized seeds were sowed on medium for plants and were cultured at 25° C. Two to three weeks after germination, hypocotyl and leaf of seedling were cut into 5 mm-1 cm long pieces with knife. Agrobacterium tumefaciens (for introducing pART27 as a control and for introducing pART27-BBEir) which had been shaking cultured for two days at 25° C. were five-fold diluted with the co-culture medium and the resulting suspensions were transferred to petri dishes, and the plant pieces were immersed in the suspensions for 10 minutes. The plant pieces were then put on Kimtowel, media were removed and the pieces were transferred to the co-culture agar media on which filter papers were put. Two days later, the plant pieces with A. tumefaciens were transferred on selection agar media (Linsmaier-Skoog media supplemented with 100 μM acetosyringone, 10 μM naphthylacetic acid, 1 μM benzyladenine and 3% sucrose). Thereafter, the plant pieces were transferred to Linsmaier-Skoog media supplemented with 200 μg/ml cefataxim, 20 μg/ml hygromycin, 10 μM naphthylacetic acid, 1 μM benzyladenine and 3% sucrose to carry out the selection. The species were transferred to fresh selection media every three weeks and healthy growing cells were selected.

Confirmation of Transformation

The presences of transgenes in the transformants were confirmed by PCR using genomic DNA.

Analysis of Alkaloids

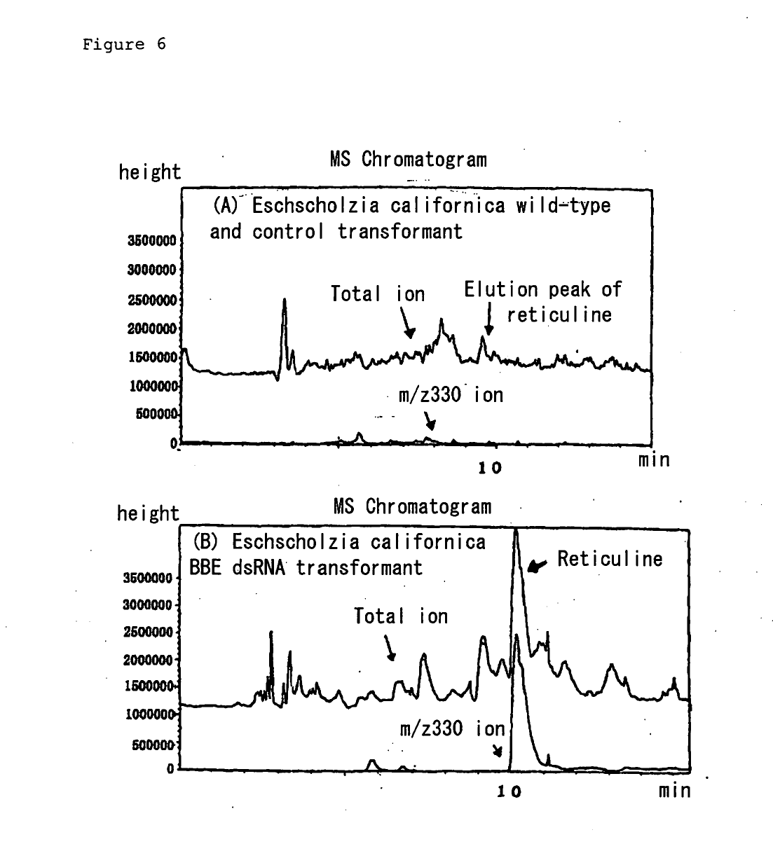

Alkaloids were extracted from the cells according to the procedure described in Proc. Nat. Acad. Sci. 98:367-372 (2001) 7. In detail, 1 g of cells was subjected to the overnight extraction with 4 ml of methanol acidified with 0.01 N HCl and the supernatant was separated by centrifugation. The extract was analyzed with Shimadzu HPLC SCL-10 system (mobile phase, 50 mM tartaric acid and 10 mM SDS/acetonitrile/methanol (4:4:1); flow rate, 1.2 ml/min; column, TSK-GEL ODS-80). Identification of each alkaloid was done with Shimadzu LC/MS-2010 system (mobile phase, water/acetonitrile/methanol/acetic acid 391:400:100:9; flow rate, 0.5 ml/min; column, TSK-GEL ODS-80) (FIG. 6).

Measurement of BBE enzyme activity

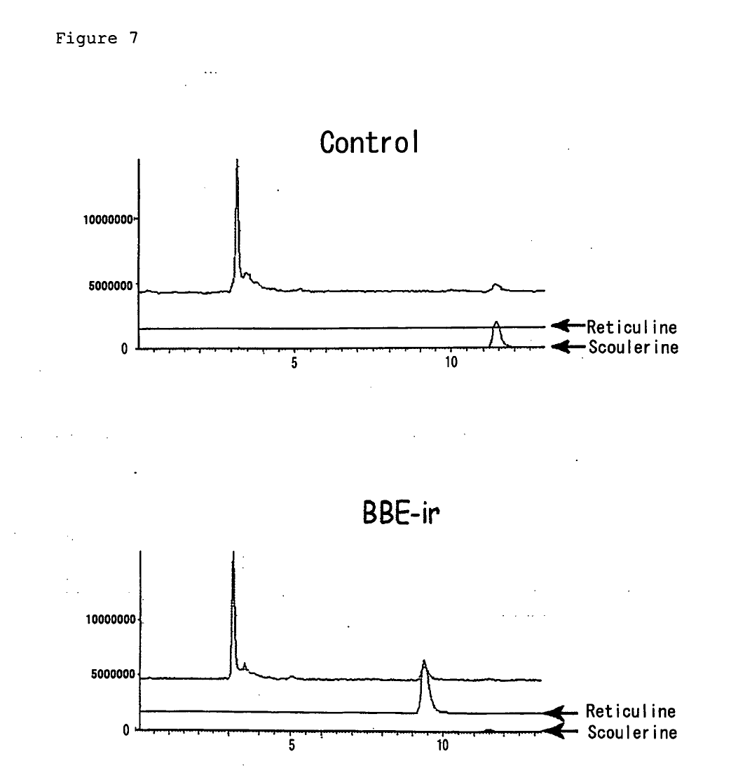

Activities of BBE of the control Eschscholzia californica and the transformant with BBE-dsRNA were measured as follows. Cultured cells (1 g-fresh weight) were added with 2 ml of glycine buffer (50 mM glycine-NaOH, pH8.9) and homogenized on ice. The extract was desalted on a PD-10 column. To 500 μl of the crude enzyme solution, the substrate reticuline was added to attain the concentration of 1 mM and the enzyme reaction was carried out at 30° C. After a predetermined period, the reaction was stopped by the addition of 10 μl of 1N NaOH. Thereafter, the production of scoulerine, the metabolite of BBE, was quantified with LC/MS (FIG. 7).

Result

With regard to Eschscholzia californica transformant which had been transformed with BBE-dsRNA, the formation of calli was observed two months after the selection. The calli were cultured in liquid media. 19 lines of controls (lines to which pART27 had been introduced) and 20 lines of transformants to which BBE-dsRNA had been introduced were obtained. There were differences of phenotypes between the controls and BBE-dsRNA transformants. The control cells were reddish while many of the BBE-dsRNA transformed cells were white. The result of HPLC analysis which examined the alkaloid composition is shown in FIG. 6. The figure indicates that reticuline was accumulated significantly in BBE dsRNA transformants (about 1.5 mg/l g-fresh weight).

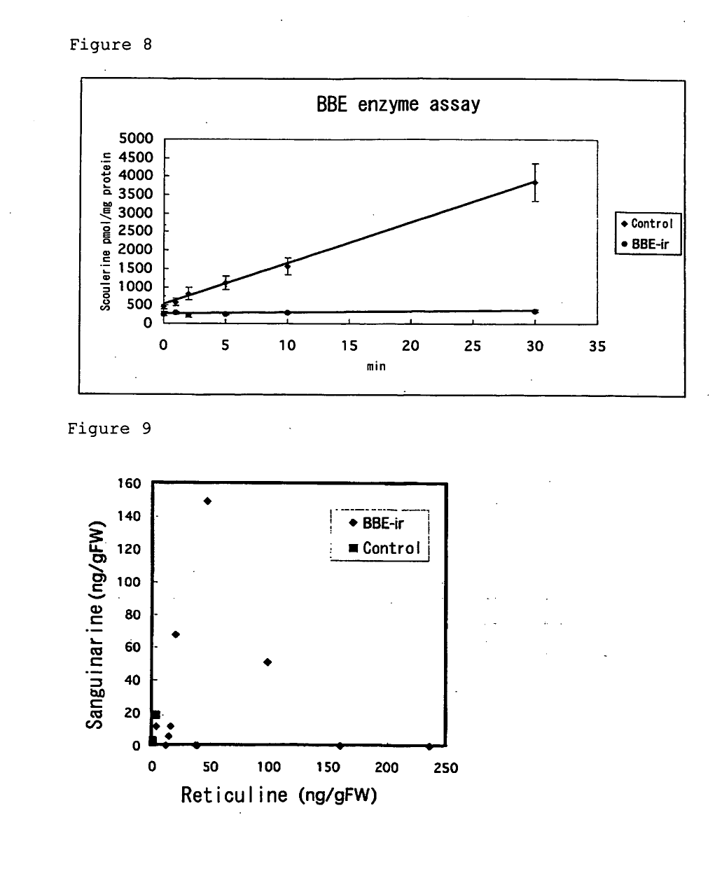

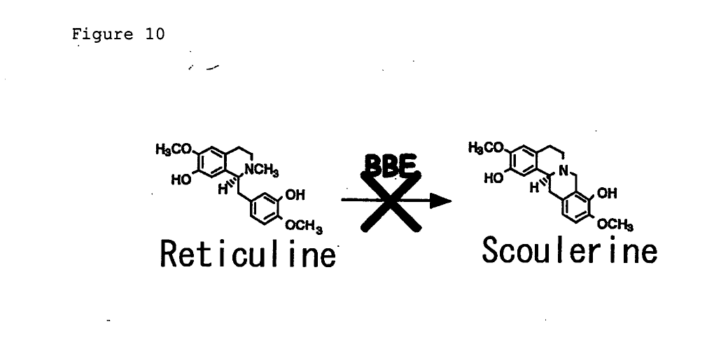

With regard to BBE enzyme activities, significant decrease in BBE activities was observed in the BBE-dsRNA transformants (called as BBEir in the Figure) compared to controls. In other words, while reticuline, the substrate of BBE, was completely converted to scoulerine in controls, there was little conversion from reticuline to scoulerine in BBE-dsRNA transformants (FIG. 7). Time-course analysis of BBE activities and quantification of the activities were carried out. As a result, while BBE activity of a control line C23 was 1.85±0.33 pkat/mg protein, that of a BBE-dsRNA transformant B14 was 0.056±0.051 pkat/mg protein. The result shows that BBE activities of the BBE-dsRNA transformants were decreased to about 3% of the controls (FIG. 8).

The contents of reticuline and sanguinarine were also determined in controls and BBE-dsRNA transformants. FIG. 9 shows that the reticuline content of BBE-dsRNA transformants were generally higher than that of controls.

On the other hand, it was reported that introduction of antisense BBE RNA expression vector into root cultures of Eschscholzia californica caused dilution of red color of the cells and considerable reduction in alkaloid contents in general. In this report, the accumulation of intermediate metabolite, which was caused by the present invention, was not observed (Plant Physiology, 128, 696-706 (2002) and Plant Molecular Biology 51:153-164 (2003)). According to the study of Plant Molecular Biology 51:153-164 (2003), by using antisense method, BBE activity of about 0.6 pkat/mg protein was remained and therefore, it is thought that the shut-off of the metabolic pathway was not sufficient.

Based on these findings, it is proved for the first time that RNAi technology is extremely effective in the shut-off of metabolic pathway and that gene silencing by RNAi technology makes it possible to suppress the metabolic reaction and causes the accumulation of intended intermediate metabolite.

The fact that the accumulation of reticuline, the substrate of BBE, was brought about by the expression of dsRNA which targeted BBE suggests that accumulation of various intermediate metabolites may be achieved by shutting-off of respective metabolic pathways.

The elucidation of alkaloid biosynthesis pathways has been developed and some enzymes involved in the biosynthesis pathways have been isolated and some of their sequences are known.

Information regarding alkaloid biosynthesis pathways may be obtained by referring, for example, P. J. Facchini, Alkaloid biosynthesis in plants: Annu. Rev. Plant Physiol. Plant Mol. Biol. 2001, 52:29-66 and KEGG:http://www.genome.ad.jp/kegg/metabolism/html. Information regarding sequences of the enzymes may be obtained by referring, for example, DDBJ:http://www.ddbj.nig.ac.jp/welcome-j.html and Genbank™:http://www/genome.ad.jp/dbget/debget.links.html.

Accordingly, other intermediates in alkaloid biosynthesis may be accumulated in plants by using the same method as that described in the present examples.

For example, with regard to isoquinoline alkaloid biosynthetic pathway shown in FIG. 1, by inhibiting norcoclaurine-6-O-methyltransferase (1) (gb:029811), coclaurine-N-methyltransferase (2) (gb:AB061863, gbu:AY217334) and coclaurine-3′-hydroxylase (3) (gb:AF014801,gb:AB025030), norcoclaurine, coclaurine and N-methylcoclaurine may be accumulated respectively. Further, with regard to indole alkaloid biosynthetic pathway shown in FIG. 2, by inhibiting glucosidase I/II (5) (gb:AF112888), strictosidine may be accumulated. The sequences of these enzymes may be obtained from the above-mentioned databanks.

### INDUSTRIAL APPLICABILITY

It is found that RNAi technology of the present invention which uses dsRNA is effective in the shut-off of metabolic pathways which produce useful compounds such as isoquinoline alkaloids. The present invention makes it possible for the first time to produce useful metabolic intermediates in the pathway. The cell lines established by the present invention may be used for development of novel biosynthetic pathways which produce novel compounds which serve as material for chemical conversion and various relevant compounds such as pharmaceutically important alkaloids.

1. A method for producing an intermediate in alkaloid biosynthesis, which comprises: inhibiting the expression of an enzyme that uses said intermediate as its substrate in an alkaloid producing plant cell, plant tissue or plant body by using RNAi technology.

2. The method according to claim 1, wherein said alkaloid is an isoquinoline alkaloid.

3. The method according to claim 1, wherein said enzyme is selected from the group consisting of berberine bridge enzyme, norcoclaurine-6-O-methyltransferase, coclaurine-N-methyltransferase and N-methylcoclaurine-3′-hydroxylase.

4. The method according to claim 3, wherein said enzyme is berberine bridge enzyme.

5. The method according to claim 1, wherein said intermediate in alkaloid biosynthesis is selected from the group consisting of reticuline, norcoclaurine, coclaurine and N-methylcoclaurine.

6. The method according to claim 5, wherein said intermediate in alkaloid biosynthesis is reticuline.

7. An intermediate in alkaloid biosynthesis produced by the method according to any one of claims 1 to 6.

8. A gene used for the method according to claim 1 which comprises:
i) a promoter, and
ii) sequences of a) and b) downstream to the promoter:
a) a forward sequence homologous to the sequence coding for all or a part of the enzyme that uses said intermediate as its substrate,
b) a reverse sequence complementary to said forward sequence.

9. A combination of genes used for the method according to claim 1 which comprises genes of A and B:
A. i) a promoter, and
ii) downstream to the promoter, a gene comprising a forward sequence homologous to the sequence coding for all or a part of the enzyme that uses said intermediate as its substrate,
B. i) a promoter, and
ii) downstream to the promoter, a gene comprising a reverse sequence complementary to said forward sequence.

10. The gene according to claim 8, wherein said enzyme is selected from the group consisting of berberine bridge enzyme, norcoclaurine-6-O-methyltransferase, coclaurine-N-methyltransferase and N-methylcoclaurine-3′-hydroxylase.

11. The combination of genes according to claim 9, wherein said enzyme is selected from the group consisting of berberine bridge enzyme, norcoclaurine-6-O-methyltransferase, coclaurine-N-methyltransferase and N-methylcoclaurine-3′-hydroxylase.

12. A vector comprising the gene according to claim 8.

13. A combination of vectors comprising;
a vector carrying the gene which comprises the forward sequence recited in claim 9, and
a vector carrying the gene which comprises the reverse sequence complementary to said forward sequence.

14. A plant cell, plant tissue or plant body, which is transformed with the vector of claim 12 or the combination of vectors of claim 13.

15. The plant cell, plant tissue or plant body according to claim 14, wherein said plant is an isoquinoline alkaloid producing plant.

16. The plant cell, plant tissue or plant body according to claim 15, wherein said plant is Eschscholzia californica.
